(19) United States
(12) Patent Application Publication
(10) Pub. No.: US 2024/0425433 A1
(43) Pub. Date: Dec. 26, 2024

(54) REACTION APPARATUS AND PRODUCTION PROCESS FOR OLEFIN PRODUCTION USING DIRECT CONVERSION OF METHANE

(71) Applicant: SOGANG UNIVERSITY RESEARCH & BUSINESS DEVELOPMENT FOUNDATION, Seoul (KR)

(72) Inventors: Kyoung-Su Ha, Seoul (KR); Won Ho Jung, Seoul (KR); Juchan Kim, Seoul (KR); Jinwon Lee, Seoul (KR)

(21) Appl. No.: 18/703,674

(22) PCT Filed: Oct. 24, 2022
(86) PCT No.: PCT/KR2022/016268

§ 371 Date: Apr. 22, 2024

(30) Foreign Application Priority Data
Oct. 22, 2021 (KR) 10-2021-0142085

(51) Int. Cl. C07C 2/80; B01J 8/00; B01J 19/08

(52) U.S. Cl. CPC C07C 2/80; B01J 8/00; B01J 19/088; B01J 2219/0898

(57) ABSTRACT

According to an embodiment of the present invention, a reaction apparatus for olefin production, including a feed separation unit that separates a methane feed and a light hydrocarbon feed from a supplied hydrocarbon feed; an ethane cracking unit that receives the light hydrocarbon feed from the feed separation unit and performs an ethane cracking process to produce an olefin product; a dielectric barrier reaction unit that receives the methane feed from the feed separation unit and generates a saturated hydrocarbon feed and an unsaturated hydrocarbon feed through plasma reaction; and a hydrogenation unit that hydrogenates at least a portion of the unsaturated hydrocarbon feed to produce an olefin product.

### TECHNICAL FIELD

The present invention relates to a production apparatus and production process for producing olefins from a carbon compound feed such as shale gas using an ethane cracking process and a plasma cracking process.

### BACKGROUND ART

Light olefins, referring to ethylene, propylene, butene, and the like, are some of the most important products in petrochemicals, and are mainly produced by pyrolysis or fluidized catalytic cracking (FCC) of naphtha. However, because of the recent depletion of petroleum resources and high petroleum prices, a number of attempts have been made to produce light olefins, a petrochemical base oil, from resources (coal, natural gas, and the like) that can replace petroleum.

Meanwhile, shale gas, which has recently attracted attention, generally consists of CH₄ (80-90% v/v) and other light alkanes (for example, C₂H₆ and C₃H₈). As with existing petroleum resources, various attempts are being made to produce light olefins from shale gas.

Currently commonly used processes are an ethane cracking process or a methane reforming process. However, these processes still have a number of problems, such as high-temperature operation and catalyst deactivation by coke generation, and the industrial use thereof is limited.

In order to industrially generate olefins from light hydrocarbon feeds, not only olefin production yield and selectivity but also the cost required to generate the same amount of oletins is greatly important. From this perspective, the existing ethane cracking process or methane reforming process is advantageous in terms of selectivity for olefin production, but has the disadvantage of poor industrial feasibility.

Therefore, extensive research and development is still needed regarding technology for industrially producing olefins from light hydrocarbon feeds.

### DISCLOSURE

### Technical Problem

An object of the present invention is to produce olefins from a hydrocarbon feed at a high yield, a high selectivity and low cost.

### Technical Solution

According to an embodiment of the present invention, there is provided a reaction apparatus for olefin production, including a feed separation unit that separates a methane feed and a light hydrocarbon feed from a supplied hydrocarbon feed; an ethane cracking unit that receives the light hydrocarbon feed from the feed separation unit and performs an ethane cracking process to produce an olefin product; a dielectric barrier reaction unit that receives the methane feed from the feed separation unit and generates a saturated hydrocarbon feed and an unsaturated hydrocarbon feed through plasma reaction; and a hydrogenation unit that hydrogenates at least a portion of the unsaturated hydrocarbon feed to produce an olefin product.

According to an embodiment of the present invention, there is provided a reaction apparatus for olefin production, in which at least one or more catalyst compounds selected from the group consisting of Al₂O₃, crystalline silica (SIO₂), and mesoporous silica (KIT-6) are provided in the dielectric barrier reaction unit, and the catalyst compound promotes generation of radicals from the methane feed and generation of the unsaturated hydrocarbon feed through a bonding reaction between radicals. However, the type of catalyst is not limited to those described above.

According to an embodiment of the present invention, there is provided a reaction apparatus for olefin production, in which a catalyst regeneration unit for regenerating the catalyst compound is further provided in the dielectric barrier reaction unit.

According to an embodiment of the present invention, there is provided a reaction apparatus for olefin production, in which the dielectric barrier reaction unit further includes a saturated hydrocarbon separation unit that separates the generated unsaturated hydrocarbon feed and saturated hydrocarbon feed from each other. The hydrocarbon teed generated here may also be supplied for an ethane cracking process.

According to an embodiment of the present invention, there is provided a reaction apparatus for olefin production, in which the hydrogenation unit separates methane contained in the unsaturated hydrocarbon feed and supplies the methane again for plasma reaction for generating an unsaturated hydrocarbon feed.

According to an embodiment of the present invention, there is provided a reaction apparatus for olefin production, in which a plurality of dielectric barrier reaction chambers is provided in the dielectric barrier reaction unit.

According to an embodiment of the present invention, there is provided an olefin production process, including a first step of separating a methane feed and a light hydrocarbon feed from a hydrocarbon feed supplied in a feed separation unit; a 2A step of supplying the light hydrocarbon feed into an ethane cracking unit and performing an ethane cracking process to produce an olefin product; a 2B step of supplying the methane feed into a dielectric barrier reaction unit and producing an unsaturated hydrocarbon feed through plasma reaction; and a third step of supplying the unsaturated hydrocarbon feed into a hydrogenation unit and producing an olefin product.

According to an embodiment of the present invention, there is provided an olefin production process, in which a saturated hydrocarbon feed and an unsaturated hydrocarbon feed are generated after the 2B step, and the unsaturated hydrocarbon feed is separated from the saturated hydrocarbon feed and supplied into the hydrogenation unit.

According to an embodiment of the present invention, there is provided an olefin production process, in which a saturated hydrocarbon feed and an unsaturated hydrocarbon feed are generated after the 2B step, and the saturated hydrocarbon feed is separated from the unsaturated hydrocarbon feed and supplied for an ethane cracking process.

According to an embodiment of the present invention, there is provided an olefin production process, in which a catalyst compound is provided inside the dielectric barrier reaction unit, and a catalyst regeneration step of regenerating the catalyst compound after plasma reaction is further performed.

According to an embodiment of the present invention, there is provided an olefin production process, in which the hydrogenation unit separates methane contained in the unsaturated hydrocarbon feed and supplies the methane again for plasma reaction for generating an unsaturated hydrocarbon feed.

### Advantageous Effects

According to an embodiment of the present invention, olefins can be produced from a hydrocarbon feed at a high yield, a high selectivity, and low cost.

According to an embodiment of the present invention, olefins can be produced from a hydrocarbon feed with less energy compared to existing processes.

### BRIEF DESCRIPTION OF DRAWINGS

FIG. 1 is a block diagram illustrating a reaction process for olefin production according to an embodiment of the present invention;

FIG. 2 is a block diagram illustrating a reaction process for olefin production according to another embodiment of the present invention;

FIG. 3 illustrates a dielectric barrier reactor according to an embodiment of the present invention;

FIG. 4 illustrates the results of analyzing the methane conversion rate and generated unsaturated hydrocarbon/saturated hydrocarbon ratio when catalyst compounds according to the Comparative Examples and the Example of the present invention are used; and

FIG. 5 illustrates the results of analyzing the generated hydrocarbon composition when catalyst compounds according to the Comparative Examples and the Example of the present invention are used.

### DETAILED DESCRIPTION OF THE INVENTION

The present invention can be subject to various changes and have various forms, and specific embodiments will be illustrated in the drawings and described in detail in the text. This is not intended to limit the present invention to a specific disclosed form, but should be understood to include all changes, equivalents, and substitutes included in the spirit and technical scope of the present invention.

In the reaction apparatus and process for olefin production according to an embodiment of the present invention, two processes (ethane cracking and plasma decomposition) for decomposing a light hydrocarbon feed having 1 to 5 carbon atoms are organically combined, and thus the yield of olefins produced from a light hydrocarbon feed is excellent and the economic feasibility of olefin production is also excellent.

FIG. 1 is a block diagram illustrating the reaction process for olefin production according to an embodiment of the present invention.

Referring to FIG. 1, the reaction apparatus/process for olefin production according to an embodiment of the present invention includes a feed separation unit 100 that separates a methane feed and a light hydrocarbon feed from a supplied hydrocarbon feed; an ethane cracking unit 300 that receives the light hydrocarbon feed from the feed separation unit 100 and performs an ethane cracking process to produce an olefin product; a dielectric barrier reaction unit 200 that receives the methane feed from the feed separation unit 100 and generates a saturated hydrocarbon feed and an unsaturated hydrocarbon feed through plasma reaction; and a hydrogenation unit 250 that hydrogenates at least a portion of the unsaturated hydrocarbon feed to produce an olefin product.

The feed separator 100 may purify/separate the hydrocarbon feed introduced into the process. Specifically, the hydrocarbon feed introduced into the feed separation unit 100 may be separated into a light hydrocarbon feed and a methane feed.

The hydrocarbon feed introduced into the feed separation unit 100 may be a mixture having a high content of alkanes, alkenes, and alkynes having 1 to 5 carbon atoms. The hydrocarbon feed may be, for example, shale gas. Shale gas may be, for example, a mixed composition having contents of methane (CH₄), ethane (C₂H₆) and propane (C₃H₈) of 80 mol %, 10 mol % and 5 mol %, respectively. In some cases, the hydrocarbon feed may contain methane (CH₄) and the like generated as by-products of other petrochemical processes.

The feed separation unit 100 may remove impurities contained in the supplied hydrocarbon feed. The feed separation unit 100 may remove, for example, impurities such as carbon dioxide (CO₂) and hydrogen sulfide (H₂S, contained in the hydrocarbon feed. In order to remove the above-mentioned impurities, the feed separation unit 100 may be provided with an aqueous amine solution tank, dust collection equipment, desulfurization equipment, and the like. However, the method for removing impurities is not particularly limited, and any means that can generally be used to remove impurities in petrochemical processes may be used. If necessary, a process of separating cycloalkanes, aromatic hydrocarbons, and the like that are unnecessary for subsequent processes may be performed in the feed separation unit 100.

The methane feed separated and generated in the teed separation unit 100 may be a mixture having a high methane content of 50% or more compared to the contents of other components. The light hydrocarbon feed may be a mixture containing hydrocarbons having 2 or 3 carbon atoms at a content of 50% or more.

As described above, the feed separation unit 100 may use methods such as distillation and pressure swing adsorption (PSA) to separate/produce a methane feed and a light hydrocarbon feed. However, in addition to the above-described methods, any method that can separate methane and hydrocarbons having 2 or 3 carbon atoms may be employed without limitation.

The methane feed separated in the feed separation unit 100 may be supplied into the dielectric barrier reaction unit 200.

The dielectric barrier reaction unit 200 receives the methane feed and generates an unsaturated hydrocarbon feed through plasma reaction. The dielectric barrier reaction unit 200 may specifically use a dielectric barrier discharge reaction. The supplied methane feed reacts with plasma in the above-described dielectric barrier discharge reaction to generate radicals such as CH₃ radicals and CH₂ radicals, and the generated radicals react with each other or with methane (CH₄) that has not yet reacted. An unsaturated hydrocarbon feed containing compounds such as ethane (C₂H₆) and propane (C₃H₈) may be generated through the radical reaction.

The dielectric barrier reaction unit 200 is different from a methane conversion reaction using conventional dielectric barrier discharge in that an unsaturated hydrocarbon feed is mainly produced from a methane feed. Among the conventional CH₄ conversion methods using DBD plasma, the most actively researched field is the dry reforming reaction of CH₄ (DRM) with CO₂ to obtain syngas or liquid products. However, this reaction process has several disadvantages, such as relatively high temperature conditions (about 673 K to about 773 K) and a high coke formation rate. In the DBD plasma reaction conducted in the dielectric barrier reaction unit 200 of the present invention, HCs including unsaturated hydrocarbons, saturated hydrocarbons, and hydrogen are mainly generated by non-oxidative conversion of methane. The composition of the material generated at this time, that is the ratio of unsaturated hydrocarbons to saturated hydrocarbons, may vary depending on the type and composition of the catalyst compound provided in the dielectric barrier reaction unit 200, and the amount and supply pattern of energy supplied.

Looking in more detail at the unsaturated hydrocarbon feed generated in the dielectric barrier reaction unit 200, the unsaturated hydrocarbon feed contains alkene compounds having a double bond as well as alkyne compounds having a triple bond. In some cases, a saturated hydrocarbon feed may also be partly generated in the dielectric barrier reaction unit 200 after plasma reaction.

The unsaturated hydrocarbon feed and saturated hydrocarbon feed generated in the dielectric barrier reaction unit 200 are separated from each other. The separated unsaturated hydrocarbon feed is supplied into the hydrogenation unit 250. The separated saturated hydrocarbon feed may be collected separately and used as an energy source or supplied into the ethane cracking unit 300.

The dielectric barrier reaction unit 200 may include various types of reactors as well as a shell-and-tube type reactor. Any reactor type is possible as long as the reactor can be manufactured with a dielectric barrier material, an electrode, and an electric circuit to generate DBD plasma at an appropriate discharged power level.

The dielectric barrier reaction unit 200 may be provided with a catalyst compound that helps radicals generated by plasma reaction to form unsaturated hydrocarbons. The catalyst compound described above may be at least one or more materials selected from the group consisting of Al₂O₃, crystalline silica (SiO₂), and mesoporous silica (KIT-6). However, the type of catalyst compound is not limited to the above examples. The catalyst compound may promote the generation of radicals from a methane feed and the generation of an unsaturated hydrocarbon feed through a bonding reaction between radicals. Since the composition of the generated material may change depending on the type of catalyst compound provided in the dielectric barrier reaction unit 200, it is important to use an appropriate catalyst compound. In other words, in the case of the present invention, the efficiency and yield of the process can be improved by minimizing the generation of saturated hydrocarbons and maximizing the generation of unsaturated hydrocarbons in the dielectric barrier reaction unit 200. Detailed analysis of changes in product composition depending on the type of catalyst compound will be described later.

The saturated hydrocarbon feed generated in the dielectric barrier reaction unit 200 is separated from the unsaturated hydrocarbon feed and supplied the hydrogenation unit 250.

The hydrogenation unit 250 hydrogenates at least a portion of the unsaturated hydrocarbon feed to produce an olefin product. Specifically, the hydrogenation unit 250 may not hydrogenate all of the unsaturated hydrocarbons contained in the unsaturated hydrocarbon feed, but may selectively hydrogenate alkyne compounds having a triple bond. Therefore, after passing through the hydrogenation unit 250, a high purity olefin product of alkene compounds having a double bond may be produced. For example, an unsaturated hydrocarbon feed containing a mixture of C₂H₂ and C₂H₄ may be supplied into the hydrogenation unit 250, and only C₂H₂ may be selectively hydrogenated to produce an oletin product having a high C₂H₄ purity.

In the hydrogenation unit 250, a process of separating alkene compounds and alkyne compounds from each other and a process of selectively hydrogenating the alkyne compounds may be performed in order to perform the above-described selective hydrogenation process. The method for separating alkene compounds and alkyne compounds from each other is not limited, and various methods such as distillation and adsorption may be used. In addition, methane, which may be contained in the unsaturated hydrocarbon feed, may also be separated in the process of separating alkene compounds and alkyne compounds from each other. The separated methane may be supplied again into the dielectric barrier reaction unit 200 for plasma reaction.

The hydrogenation unit 250 may be further provided with a hydrogenation catalyst compound such as Pd/α-Al₂O₃ for hydrogenation of alkyne compounds. At this time, in a case where the alkyne compound is excessively hydrogenated, a saturated hydrocarbon compound may be provided, so the catalyst compound allows the alkyne compound to be selectively hydrogenated into an alkene compound. However, the hydrogenation catalyst compounds described above are merely examples, and other hydrogenation catalyst compounds may also be used.

In the hydrogenation unit 250, an olefin compound is produced through the above-described separation of alkene compounds and alkyne compounds from each other and selective hydrogenation of the alkyne compounds.

Next, the ethane cracking unit 300 performs an ethane cracking process on the light hydrocarbon feed supplied from the feed separation unit 100 to produce an oletin product. At this time, as described above, the saturated hydrocarbon feed selectively produced and separated in the dielectric barrier reaction unit 200 may be supplied into the ethane cracking unit 300.

A tubular reactor may be used in the ethane cracking unit 300. In a tubular reactor, the residence time of the feed gas in the reactor may determine the production distribution. When the residence time of the reactants in the ethane cracking unit 300 is too short, the reaction may not sufficiently take place and the olefin yield may decrease. When the residence time is too long, the CH₄ selectivity is high and the olefin yield may be low. Therefore, the residence time of reactants may be adjusted by considering the amount of saturated hydrocarbon feed supplied into the ethane cracking unit 300.

In the present invention, olefin compounds may be produced in the ethane cracking unit 300, and at the same time, olefin compounds may also be produced using the dielectric barrier reaction unit 200 and hydrogenation unit 250. Accordingly, the supplied hydrocarbon feed is used more complexly for the production of olefin compounds, and thus the olefin compound production yield may be greatly improved.

As described above, according to an embodiment of the present invention, a dielectric barrier reaction unit and an ethane cracking unit are organically combined to process a hydrocarbon feed, and thus a greater amount of olefin compounds can be produced from the hydrocarbon feed.

In addition, according to an embodiment of the present invention, a reaction process for olefin production using the above-described reaction apparatus can be performed. According to an embodiment of the present invention, an olefin production process may be provided, which includes a first step of separating a methane feed and a light hydrocarbon feed from a hydrocarbon feed supplied in a feed separation unit; a 2A step of supplying the light hydrocarbon teed into an ethane cracking unit and performing an ethane cracking process to produce an olefin product; a 2B step of supplying the methane feed into a dielectric barrier reaction unit and producing an unsaturated hydrocarbon feed through plasma reaction; and a third step of supplying the unsaturated hydrocarbon feed into a hydrogenation unit and producing an olefin product.

The reaction apparatus/process for olefin production of the present invention may further include various additional configurations to achieve the purpose of improving the olefin production efficiency and reducing the process cost.

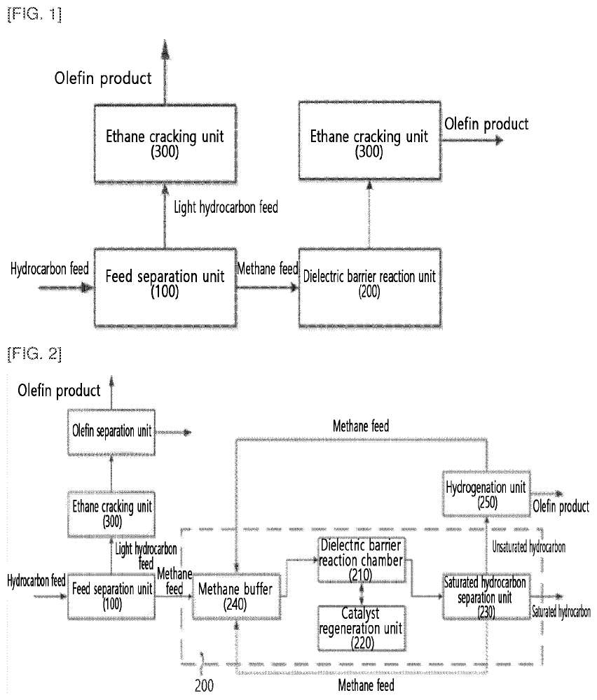

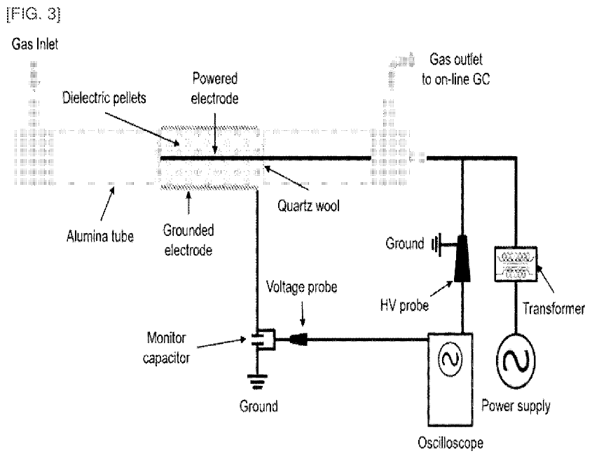

FIG. 2 is a block diagram illustrating the reaction process for olefin production according to another embodiment of the present invention.

Referring to FIG. 2, the dielectric barrier reaction unit 200 further includes a dielectric barrier reaction chamber 210, a catalyst regeneration unit 220, a saturated hydrocarbon separation unit 230, and a methane buffer 240, and an olefin separation unit 310 is further provided at the rear of the ethane cracking unit 300.

The dielectric barrier reaction chamber 210 refers to a reactor in which the plasma reaction according to dielectric barrier discharge (DBD) is conducted. The dielectric barrier reaction chamber 210, which is a reactor, may be provided with a dielectric barrier material, an electrode, and an electric circuit to generate DBD plasma at an appropriate discharged power level. The dielectric barrier reaction chamber 210 including the above-described members may be in various forms such as a tubular reactor as previously discussed as well as a shell-and-tube reactor, a multiple-rod reactor, and a multiple stacked cell-type reactor.

A plurality of dielectric barrier reaction chambers 210 may be provided. The plurality of dielectric barrier reaction chambers 210 may be operated simultaneously or alternatively. For example, when the amount of introduced methane feed is large, the dielectric barrier reaction chambers 210 may be operated simultaneously to increase the methane feed throughput per hour. In some cases, the plurality of dielectric barrier reaction chambers 210 may be operated alternatively. At this time, a catalyst regeneration process to regenerate the catalyst compound may be performed in the dielectric barrier reaction chamber 210 that is not in operation. After catalyst regeneration is completed, the dielectric barrier reaction chamber 210 that has been in operation may enter an idle state and catalyst regeneration may be performed. By alternatively operating the plurality of dielectric barrier reaction chambers 210 and performing catalyst regeneration in this way, the lifespan of catalyst compound may increase and a high level of reaction efficiency may be maintained even during continuous processes.

For the above-described catalyst regeneration operation, a catalyst regeneration unit 220 is provided.

The catalyst regeneration unit 220 may perform operations to remove coke adsorbed on the catalyst compound provided inside the dielectric barrier reaction chamber 210, and the like. The catalyst regeneration unit regenerates the catalyst compound by providing air flow inside the dielectric barrier reaction chamber 210. The catalyst regeneration unit 220 may be connected to a plurality of dielectric barrier reaction chambers 210, and when it is determined that regeneration of the catalyst compound is necessary, the plasma reaction of the methane feed in the dielectric barrier reaction chamber 210 may be stopped and catalyst regeneration may be performed by providing high-temperature air inside the dielectric barrier reaction chamber 210.

Next, the saturated hydrocarbon separation unit 230 may separate the unsaturated hydrocarbon feed and saturated hydrocarbon feed generated after plasma reaction from the dielectric barrier reaction chamber 210. The saturated hydrocarbon separation unit 230 may perform the above-described separation through methods such as distillation and pressure swing adsorption (PSA).

The saturated hydrocarbon feed separated in the saturated hydrocarbon separation unit 230 mainly contains ethane and propane, and these may be collected separately and used for energy production or moved to the ethane cracking unit 300 to be used in the ethane cracking reaction as described above. In comparison, the unsaturated hydrocarbon feed separated from the saturated hydrocarbon feed in the saturated hydrocarbon separation unit 230 is moved to the hydrogenation unit 250 and used to produce olefin compounds. In addition, the unsaturated hydrocarbon feed separated at this time may contain methane. Methane is separated from the alkene and alkyne compounds in the hydrogenation unit and then transferred to the methane buffer 240 to be used again in the dielectric barrier reaction. Therefore, even if the plasma reaction inside the dielectric barrier reaction chamber 210 is not complete, unreacted methane continues to be used in the plasma reaction, and the process yield of ultimately generating a saturated hydrocarbon feed from methane is excellent. However, in some cases, methane may be separated in the saturated hydrocarbon separation unit 230. Therefore, in this case, the methane feed, saturated hydrocarbon feed, and unsaturated hydrocarbon feed may be separated in the saturated hydrocarbon separation unit 230, and each may be transferred/used in different directions.

As described above, the methane buffer 240 functions to mix the methane feed separated and returned from the hydrogenation unit 250 or saturated hydrocarbon separation unit 230 and the methane feed provided from the feed separation unit 100. In some cases, a process of removing impurities that may affect the plasma reaction may be additionally performed in the methane buffer 240.

Next, an olefin separation unit 310 may be further provided at the rear of the ethane cracking unit 300. The olefin separation unit 310 may separate olefin compounds generated after the ethane cracking reaction from other compounds. For example, only ethylene and propylene may be utilized as olefin compounds, and other hydrocarbon compounds may be separated and utilized as energy sources.

The method for separating olefin compounds in the olefin separation unit 310 is not limited. For example, separation of olefin compounds may be performed through methods such as distillation and pressure swing adsorption (PSA).

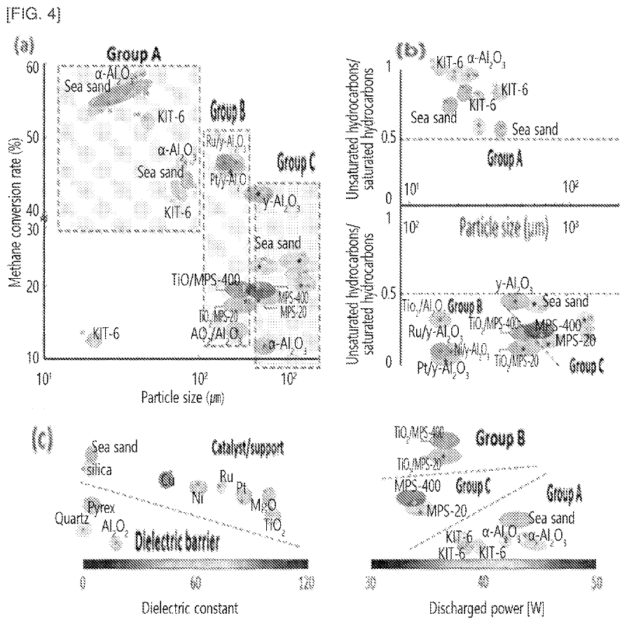

FIG. 3 illustrates the dielectric barrier reactor according to an embodiment of the present invention.

The dielectric barrier reactor according to FIG. 3 may include a powered electrode, a grounded electrode, dielectric pellets used as a dielectric barrier, a gas inlet, and a gas outlet. The powered electrode may be connected to a power supply unit that supplies power to generate plasma.

FIG. 3 illustrates a tubular reactor form in which the above-described members are provided in a pipe, and reactants and products flow in and out through the pipe. However, as previously discussed, the form of the dielectric barrier reactor is not limited to the above-mentioned examples. In addition to the above-mentioned forms, the dielectric barrier reactor may be provided in various forms such as a multiple-rod reactor and a multiple stacked cell-type reactor.

According to embodiments, the reaction in the dielectric barrier reactor may be conducted at room temperature and atmospheric pressure. In Examples, a methane mixture (CH₄:N₂=1:1) having a space velocity (SV) of 750 h⁻¹ to 1500 h⁻¹ was injected into the reactor during the reaction, and the total reaction time was about 380 minutes. In Examples, an alumina tube having an inner diameter of 6 mm and an outer diameter of 10 mm was used as a tubular reactor in the system. A stainless steel bar having a diameter of 3 mm and a length of 50 mm was used as the powered electrode, and a steel net wrapped with a steel wire was used as the grounded electrode. At this time, 150 mm of plasma bed was wrapped around the grounded electrode. The discharge gap between the inner surface of the alumina tube and the powered electrode was 1.5 mm. Therefore, the volume of this plasma emission region was fixed at 3.18 cm³. In Examples, the plasma bed was provided with as-prepared sea sand particles having two particle size ranges (0.2 mm to 1.0 mm and 1.0 mm to 1.2 mm). A sinusoidal power supply device (0 V to 220 V, 60 Hz to 1000 Hz) was connected to a transformer (0 kV to 20 kV, 1000 Hz) to supply sinusoidal AC power to the plasma bed. The applied voltage and frequency were set to 15 kV (equivalent to 30 KV peak-to-peak voltage) and 1 kHz, respectively.

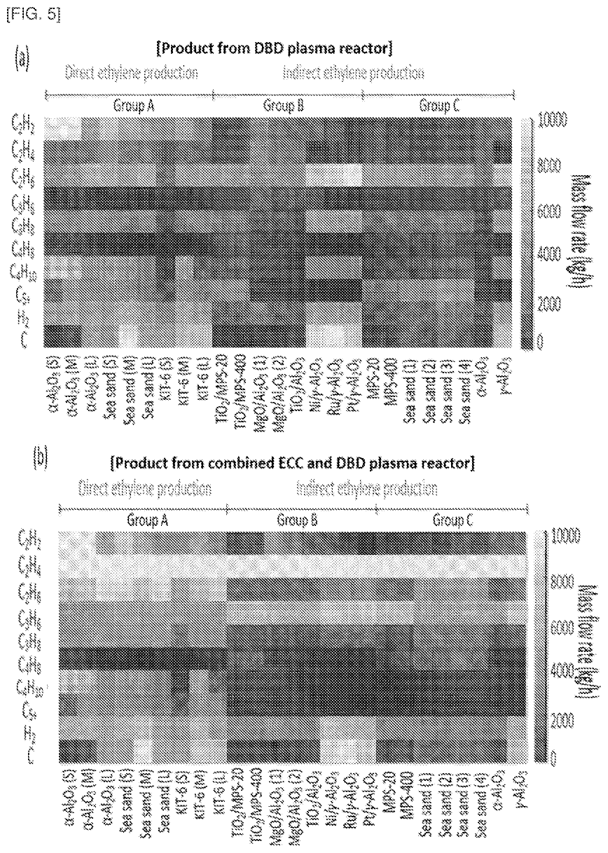

FIG. 4 illustrates the results of analyzing the methane conversion rate and generated unsaturated hydrocarbon/saturated hydrocarbon ratio when catalyst compounds according to the Comparative Examples and the Example of the present invention are used.

The composition distribution of the product in the dielectric barrier reaction unit is influenced by factors such as dielectric particle size, dielectric constant, catalyst particle size, and metal impregnation.

The catalyst particle size had the greatest influence on product distribution. As the catalyst particle size increased, the CH₄ conversion rate decreased (A of FIG. 4). When a catalyst having a particle size of 100 μm was used, the CH₄ conversion rate was about 40% to about 60%, and the ratio of unsaturated hydrocarbons to saturated hydrocarbons was higher than 0.5 as illustrated in B of FIG. 4. When the above-described catalyst was used, acetylene exhibited the highest yield and a small amount of ethylene was also generated.

Conversely, when the catalyst particle size was greater than 100 μm, the CH₄ conversion rate was relatively low (about 10% to about 50%). At this time, the ratio of unsaturated hydrocarbons to saturated hydrocarbons was less than 0.5. In this case, the yield of saturated hydrocarbons consisting mainly of C₂H₆, C₃H₈ and C4+ was high. This region includes both non-metallic dielectric particles and metal-impregnated dielectric catalysts having different dielectric properties.

In the present invention, dielectric catalyst species were classified into small non-metal dielectric particles (Group A) of Example, large dielectric particles loaded with a metal or metal oxide (Group B) of the Comparative Examples, and large non-metal dielectric particles (Group C) of the Comparative Examples depending on the particle size and presence or absence of metal impregnation.

In the case of catalyst compounds of the present invention, as can be seen from Table 1 below, it has been confirmed that excellent effects are exhibited when the particle diameter is in the range of about 25 μm to about 100 μm.

TABLE 1

| | | | X_{CH4} | Unsat. | Sat. | H₂ | V | f |
|---|---|---|---|---|---|---|---|---|
| | Catalyst | dₚ [μm] | [%] | HCs [%] | HCs [%] | [%] | [kV] | [kHz] |
| Group A | α-Al₂O₃ (S) 1 | 25 | 54.8 | 53.5 | 46.5 | 59.00 | 15 | 1 |
| (Example) | α-Al₂O₃ (M) 1 | 50 | 58.4 | 49.1 | 48.8 | 58.00 | 15 | 1 |
| | α-Al₂O₃ (L) 1 | 100 | 46.4 | 27.5 | 53.8 | 50.00 | 15 | 1 |
| | Sea sand (S) 1 | 25 | 54.6 | 38.2 | 48.1 | 64 | 15 | 1 |
| | Sea sand (M) 1 | 50 | 57.2 | 22.2 | 42.1 | 65 | 15 | 1 |
| | Sea sand (L) 1 | 100 | 43.5 | 21.9 | 53.8 | 52 | 15 | 1 |
| | KIT-6 (S) 1 | 25 | 22.5 | 29.7 | 29.9 | 59 | 15 | 1 |
| | KIT-6 (M) 1 | 50 | 52.9 | 23.6 | 44.7 | 59.5 | 15 | 1 |
| | KIT-6 (L) 1 | 100 | 41.8 | 16.0 | 44.3 | 52 | 15 | 1 |
| Group B | TiO₂/MPS-20 2 | 350 | 18.63 | 15.1 | 83.8 | 48.60 | 15 | 1 |
| (Comparative | TiO₂/MPS-400 2 | 350 | 19.96 | 16.7 | 83.1 | 46.63 | 15 | 1 |
| Examples) | MgO/Al₂O₃ (1) 3 | 250 | 23.0 | 68 | 32.0 | 51.58 | 6 | 3 |
| | MgO/Al₂O₃ (2) 3 | 500 | 16.2 | 55.7 | 44.3 | 52.04 | 6 | 3 |
| | TiO₂/Al₂O₃ 3 | 500 | 14.0 | 49.0 | 45.0 | 46.80 | 6 | 3 |
| | Ni/γ-Al₂O₃ 4 | 200 | 48.1 | 7.54 | 52.2 | 38.89 | 3, 3.2, 4 | 23 |
| | Ru/γ-Al₂O₃ 4 | 200 | 48.1 | 2.65 | 54.1 | 33.55 | 3, 3.2, 4 | 23 |
| | Pt/γ-Al₂O₃ 4 | 200 | 47 | 0 | 62.7 | 32.92 | 3, 3.2, 4 | 23 |
| Group C | MPS-20 2 | 350 | 18.59 | 17.6 | 79.3 | 49.62 | 15 | 1 |
| (Comparative | MPS-400 2 | 350 | 20.61 | 16.4 | 78.1 | 46.09 | 15 | 1 |
| Examples) | Sea Sand (1) a | 600 | 24.97 | 18.99 | 60.48 | 41.92 | 15 | 1 |
| | Sea Sand (2) a | 1100 | 23.35 | 12.85 | 68.16 | 43.45 | 15 | 1 |
| | Sea Sand (3) a | 1100 | 16.57 | 15.59 | 64.98 | 39.94 | 15 | 1 |
| | Sea Sand (4) a | 1100 | 12.54 | 17.59 | 63.17 | 39.22 | 15 | 1 |
| | α-Al₂O₃ 3 | 500 | 10.0 | 59.0 | 37.0 | 50.28 | 6 | 3 |
| | γ-Al₂O₃ 4 | 200 | 42.5 | 10.31 | 53.4 | 39.69 | 3, 3.2, 4 | 23 |

As can be seen from the figure, the proportion of unsaturated hydrocarbons generated after plasma reaction was high in the case of Group A of Example, and the proportion of saturated hydrocarbons generated was high in the case of Group B and Group C of the Comparative Examples. Therefore, it can be seen that when the catalyst compounds of Group A are used, the proportion of unsaturated hydrocarbons is high and olefin products can be produced directly by selectively hydrogenating the unsaturated hydrocarbons. In comparison, it can be seen that when the catalyst compounds of Group B and Group C of the Comparative Examples are used, the proportion of saturated hydrocarbons is high, and it is unsuitable to directly produce olefin products by hydrogenating the saturated hydrocarbons.

In order to further validate the feasibility of commercialization. CH₄ conversion experiments were conducted using the catalyst compounds of each group. The dielectric constants of catalyst compound particles of the three groups are illustrated in C of FIG. 4.

The materials mainly used as dielectric barriers when dielectric barrier plasma reaction is conducted, are alumina, silica, and Pyrex tubes, which have dielectric constants of 9.8, 3.9, and 4.84, respectively. In general, the dielectric constants of metals or metal oxides are higher than those of alumina, silica, and Pyrex.

From the experimental results illustrated in C of FIG. 4, it can be seen that the methane conversion reaction can be conducted when the catalyst compounds of the Example are used as well.

The distribution and heat map of products from the dielectric barrier reaction unit using DBD plasma reaction and the combined processes with the ethane cracking unit and the dielectric barrier reaction unit are illustrated in A and B of FIG. 5, respectively. Referring to A of FIG. 5, the C₂H₂ fraction generated after plasma reaction was the largest in Group A, the catalysts of Example. In Example, C₂H₄, C₂H₆, C₄H₁₀, and H₂ were produced in large quantities, whereas C₃H₆ and C₄H₈ were produced in small quantities. Therefore, it can be seen that when the catalysts of the Example are utilized, C₂H₄, an olefin product can be directly produced by hydrogenating the generated C₂H₂, and it can be seen that there are few reaction by-products and the reaction selectivity is high since the amount of C₃H₆ and C₄H₈ generated is small.

The catalysts of Group B and Group C of the Comparative Examples showed similar product distributions. In Group B, there was a difference in the generation of C₂H₄ and coke deposits, and it was found that the catalytic functions of the metal oxides and metals contained in the catalyst compounds of Group B affected the plasma reaction. More specifically, dielectric catalysts impregnated with metals, such as Ni, Ru, and Pt, among the catalyst compounds of Group B can generate more saturated hydrocarbons than olefins by hydrogenation. Therefore, it can be seen that when these catalysts are used, it is difficult to produce olefin compounds through hydrogenation as in the present invention.

Next, in the case of a combined processes with the ethane cracking unit and the dielectric barrier reaction unit, as can be seen from B of FIG. 5, a large amount of ethylene was generated in all catalyst groups of Examples and the Comparative Examples through the ethane cracking process. The product distribution of Group A, catalysts of Example, is a mixture of alkyne compounds and alkene compounds, but the amount of C₂H₄ may be increased by the hydrogenation of C₂H₂. In Groups B and C of the Comparative Examples, the amount of C₃H₆ generated significantly increased. In the case of using catalyst compounds of the Comparative Examples, the selectivity for other hydrocarbons except for C₂H₄ and C₃H₆ was very low because of the consumption of C4 and C5+ in the ethane cracking unit. Therefore, it can be seen that when the catalyst compounds of the Comparative Examples are used, it is difficult to directly produce ethylene or produce ethylene through hydrogenation of acetylene.

Next, the compositions of reaction products in the ethane cracking unit used in the process together with the dielectric barrier reaction unit were analyzed. When a pure C₂H₆ feed is supplied into the ethane cracking unit, a C₂H₆—C₃H₈ mixture is supplied, and naphtha is supplied, the reaction product compositions are as shown in the table below.

TABLE 2

| Composition | C₂H₆ | C₂H₆-C₃H₈ | |
|---|---|---|---|
| (wt. %) | feed | feed | Naphtha feed |
| H2 | 3.77 | 3.1 | 1.0 |
| CH4 | 3.90 | 8.5 | 16.6 |
| C2H2 | 0.43 | 0.1 | 0 |
| C2H4 | 47.40 | 44.1 | 30.8 |
| C2H6 | 39.48 | 26.5 | 4.0 |
| C3H6 | 0.50 | 9.9 | 13.9 |
| C3H8 | 0.15 | 4.2 | 0 |
| C4H6 | 0 | 0 | 4.5 |
| C4H8 | 0 | 0 | 5.0 |
| Aromatics | 0 | 0 | 24.2 |

As the proportion of C₂H₆ in the feed to the ethane cracking unit increased, the selectivity for ethylene increased, whereas the selectivity for by-products such as C4+ hydrocarbons converged to almost 0. Conversely, in a case where C5+ contained in naphtha was supplied, the C₂H₄ yield decreased, whereas the C₃H₆ and C₄H₈ yields increased.

In the existing ethane cracking process, the production yield of olefins including ethylene from shale gas containing CH₄, C₂H₆, and C₃H₈ was only about 20.8%. However, when the combined processes according to an embodiment of the present invention is used, the olefin production yield increases to about 24.5% to about 38.3%, in addition, in a case where 90% of the unreacted CH₄ is recycled in the dielectric barrier reaction unit, the olefin production yield increases to about 45.9% to about 60.4%.

Consequently, it means that the combined processes according to an embodiment of the present invention can sufficiently secure the yield and economic feasibility compared to the existing process.

In the above, the present invention has been described with reference to preferred embodiments, but those skilled in the art or those having ordinary knowledge in the art will understand that various modifications and changes can be made to the present invention without departing from the spirit and technical scope of the present invention as set forth in the claims to be described later.

Therefore, the technical scope of the present invention should not be limited to what is described in the detailed description of the specification, but should be defined by the claims.

1. A reaction apparatus for olefin production, comprising:
a feed separation unit that separates a methane feed and a light hydrocarbon feed from a supplied hydrocarbon feed;
an ethane cracking unit that receives the light hydrocarbon feed from the feed separation unit and performs an ethane cracking process to produce an olefin product;
a dielectric barrier reaction unit that receives the methane feed from the feed separation unit and generates a saturated hydrocarbon feed and an unsaturated hydrocarbon feed through plasma reaction; and
a hydrogenation unit that hydrogenates at least a portion of the unsaturated hydrocarbon feed to produce an olefin product.

2. The reaction apparatus for olefin production according to claim 1, wherein
at least one or more catalyst compounds selected from the group consisting of Al₂O₃, crystalline silica (SiO₂), and mesoporous silica (KIT-6) are provided in the dielectric barrier reaction unit, and
the catalyst compound promotes generation of radicals from the methane feed and generation of the unsaturated hydrocarbon feed through a bonding reaction between radicals.

3. The reaction apparatus for olefin production according to claim 2, wherein
a catalyst regeneration unit for regenerating the catalyst compound is further provided in the dielectric barrier reaction unit.

4. The reaction apparatus for olefin production according to claim 1, wherein
the dielectric barrier reaction unit further includes a saturated hydrocarbon separation unit that separates the generated unsaturated hydrocarbon feed and saturated hydrocarbon feed from each other.

5. The reaction apparatus for olefin production according to claim 1, wherein
the hydrogenation unit separates methane contained in the unsaturated hydrocarbon feed and supplies the methane again for plasma reaction for generating an unsaturated hydrocarbon feed.

6. The reaction apparatus for olefin production according to claim 1, wherein
a plurality of dielectric barrier reaction chambers is provided in the dielectric barrier reaction unit.

7. An olefin production process comprising:
a first step of separating a methane feed and a light hydrocarbon feed from a hydrocarbon feed supplied in a feed separation unit;
a 2A step of supplying the light hydrocarbon feed into an ethane cracking unit and performing an ethane cracking process to produce an olefin product;
a 2B step of supplying the methane feed into a dielectric barrier reaction unit and producing an unsaturated hydrocarbon feed through plasma reaction; and
a third step of supplying the unsaturated hydrocarbon feed into a hydrogenation unit and producing an olefin product.

8. The olefin production process according to claim 7, wherein
a saturated hydrocarbon feed and an unsaturated hydrocarbon feed are generated after the 2B step, and the unsaturated hydrocarbon feed is separated from the saturated hydrocarbon feed and supplied into the hydrogenation unit.

9. The olefin production process according to claim 7, wherein
a saturated hydrocarbon feed and an unsaturated hydrocarbon feed are generated after the 2B step, and the saturated hydrocarbon feed is separated from the unsaturated hydrocarbon feed and supplied for an ethane cracking process.

10. The olefin production process according to claim 7, wherein
a catalyst compound is provided inside the dielectric barrier reaction unit, and
a catalyst regeneration step of regenerating the catalyst compound after plasma reaction is further performed.

11. The olefin production process according to claim 7, wherein
the hydrogenation unit separates methane contained in the unsaturated hydrocarbon feed and supplies the methane again for plasma reaction for generating an unsaturated hydrocarbon feed.
